(19) United States
(12) Patent Application Publication
(10) Pub. No.: US 2025/0023728 A1
(43) Pub. Date: Jan. 16, 2025

(54) Secure BYOD Programmer

(71) Applicant: BIOTRONIK SE & Co. KG, Berlin (DE)

(72) Inventors: Jens MUELLER, Berlin (DE); Thomas DOERR, Berlin (DE); Miro SELENT, Nuthetal (DE)

(73) Assignee: BIOTRONIK SE & Co. KG, Berlin (DE)

(21) Appl. No.: 18/710,237

(22) PCT Filed: Nov. 10, 2022
(86) PCT No.: PCT/EP2022/081415

§ 371 Date: May 15, 2024

(30) Foreign Application Priority Data
Nov. 19, 2021 (EP) 21209313.2

(51) Int. Cl. H04L 9/14; G16H 40/67

(52) U.S. Cl. CPC H04L 9/14; G16H 40/67; H04L 2209/88

(57) ABSTRACT

Methods and systems for securely communicating with an implant. A method of communication by a local communication system, with an implant and with a remote communication system may comprise the steps of: receiving a user input indicative of first data to be transmitted to the implant, transmitting a first indication associated with the user input to the remote communication system for processing, prior to sending the first data to the implant. Additionally or alternatively, the method may comprise receiving second data from the implant, and, without accessing the second data, transmitting a second indication associated with the second data to the remote communication system for processing.

### CROSS-REFERENCE TO RELATED APPLICATIONS

This application is the United States National Phase under 35 U.S.C. § 371 of PCT International Patent Application No. PCT/EP2022/081415, filed on Nov. 10, 2022, which claims the benefit of European Patent Application No. 21209313.2, filed on Nov. 19, 2021, the disclosures of which are hereby incorporated by reference herein in their entireties.

### TECHNICAL FIELD

The present invention relates to methods and systems for (securely) communicating with an implant.

### BACKGROUND

State-of-the-art implants (such as, e.g., pacemakers, defibrillators, etc.) provide the opportunity of programming the implant such that individual needs of a patient may be met. Such implants, when implemented as, e.g., a pacemaker, may provide several operation modes each being capable of addressing, e.g., a different heart related disease. Such implants may be implanted and adapted to a current medical situation of the patient during the implantation and may provide the option of readjusting and/or finetuning the implant even years after the implantation such that the implant may be adjusted to a changing health situation of the patient over time.

It is typical that specific and mostly proprietary programming devices are used for communication (e.g., interrogation, programming, etc.) with the implant. Such devices are oftentimes supplied by the manufacturer of the implant and/or associated third party suppliers. However, this comes with the drawback of increased costs for the programming devices, work-flow disturbances (e.g., in a hospital) if such a device shows a defective behavior, generically a limited number of available devices, dedicated and complicated maintenance requirements (e.g., for the installation of software updates), etc. It may therefore be seen as beneficial to use bring-your-own-device (BYOD)-based solutions for, e.g., programming an implant such as, e.g., commercially available consumer products (e.g., arbitrary smartphones, tablets, etc. without being manufactured for a dedicated purpose or a specific manufacturer). However, these devices are typically not adapted and/or specialized for communicating with implants in a sufficiently reliable manner. This particularly applies for communication with life-surveilling and life-saving implants (such as class III devices). Using BYOD devices that may easily be subject to hacker attacks may result in a life threating risk for the patient. In other words, standard BYOD-based solutions are inherently regarded as unsecure with respect to the communication with an implant and difficult to administrate. In particular, such devices may comprise security related weak spots (e.g., through third party apps) and may be subject to the installation of malware, criminal motivated remote control (e.g., by means of trojans), may suffer from a lack of regular security updates (e.g., as no dedicated staff may be entrusted to account for regular updates on the BYODs), may comprise manipulated right management for user accounts on the BYODs and/or specific apps, etc. which may allow an undesired manipulation of the data to the be sent to the implant and/or a tapping of data sent between the implant and the BYOD. Even if an encrypted communication method is used, the encryption may nevertheless be subject to hacker attacks if, e.g., malware on the BYOD-based solution affects the encryption.

There are approaches to address drawbacks of BYOD devices by tools to secure apps from reverse engineering attempts, wherein reverse engineering may be used to change the internal procedures of apps with the effect of rendering these apps unsecure. For example, a processing logic (e.g., a programmer app) for programming and interrogating of implants may be performed locally on a BYOD, and it may be equipped with obfuscation mechanisms to prevent reverse engineering attacks. The establishing of a connection may occur by means of a mediator (e.g., a coil/Bluetooth low energy (BLE) converter) in between the implant and the programmer app. The implant may be woken up by the mediator and a secret may be exchanged between the mediator and the implant as a measure to protect the subsequent communication. However, these measures do not account for any remote manipulation of the processing logic on the BYOD which may then be decrypted or operated remotely. It may thus still be possible to transmit malicious software onto the implant, for example.

In short, also precautions related to reverse engineering do not provide sufficient protection for BYODs which are used for communication with implants, in particular class III devices, as a remote and/or a local manipulation and/or operation of BYODs may not fully be excluded. Such a manipulation may, e.g., relate to a decryption of the communication logic with the BYOD (e.g., relating to the programming of implants and/or the interrogation of implants) which may facilitate intrusion attacks of malicious software and/or scripts onto the BYOD and onto the implant.

The present disclosure is directed toward overcoming one or more of the above-mentioned problems, though not necessarily limited to embodiments that do.

### SUMMARY

Therefore, there is a need for allowing the use of BYOD devices as programming devices for implants, however, without introducing any safety risks and without disturbing the workflow of, e.g., the medical staff interacting with the programming devices. According to an aspect of the present invention, this need is at least partly met by a method for communication, by a local communication system, with an implant and with a remote communication system. The method may comprise the steps of: receiving a user input indicative of first data to be transmitted to the implant and transmitting a first indication associated with the user input to the remote communication system for processing, prior to sending the first data to the implant.

Requiring a first indication associated with the first data to be transmitted to a third entity (e.g., acting as a backend infrastructure), namely the remote communication system, which may be a securely administrated communication entity (e.g., by the manufacturer of the implant or any other third party which is proven as trustful), prior to sending the first data to the implant, may ensure a secure communication between the local communication system and the implant. For example, the indication may indicate the first data and/or comprise at least a portion of the first data and/or it may comprise additional information associated with the local communication system, the remote communication system and/or the implant (e.g., such as a system specific identifier, a check-sum, etc.). It may alternatively or additionally be possible that the first indication only comprises information associated with the user input, such as, e.g., information that a certain user input has been performed (e.g., pressing of a button, a spoken sequence, etc.). The remote communication system may securely process the first indication (e.g., carrying out a validity check, encrypting data, decrypting data, etc.), and may thus verify whether the first indication associated with the user input indicates first data that is valid for the implant (e.g., within certain parameter ranges, plausible, comprises authorized commands, etc.) and/or whether the local communication system is entitled to send the first data to the implant. Thus, for example, in case the local communication system is hacked, the remote communication system may realize this, based on the notification, and may prevent the local communication system from sending the first data to the implant, e.g., by not issuing a corresponding authorization, not conveying the first data to the local communication system, etc. This may avoid that unauthorized third party devices interact with the implant and/or that data (e.g., programming settings) unsuitable for the implant may be sent to the implant that may deteriorate the patient state. In other words, the remote communication system may act as a gatekeeper that secures the access to the implant.

A local communication system may relate to at least one BYOD. Such a BYOD may comprise consumer electronics such as, e.g., a smartphone, a tablet, a smartwatch, virtual reality glasses, etc., e.g., without specific firmware and/or other customization other than that may, e.g., provided by an app that may be downloaded to the BYOD. Additionally or alternatively, it may also be possible that the BYOD relates to a computer with a respective interface (e.g., dongle) which facilitates a communication of the computer with the implant. Additionally or alternatively, it may also be possible that the BYOD comprises smart devices such as, e.g., a smartspeaker, a smart TV, etc. Additionally or alternatively, it may be possible that the local communication system comprises an off-the-shelf device manufactured by a manufacturer of medical implants (and/or administrators of a corresponding remote communication system) which may adopt the secure communication as disclosed herein. By means of at least the aspects described herein, a secure communication with an implant may be provided by any such device.

The first data to be sent to the implant may relate to data associated with an interrogation of the implant (e.g., a request for certain operation parameters of the implant (e.g., the current battery status), the current pulse rate of the patient, etc.) and/or at least one programming command to be sent to the implant (e.g., associated with setting the implant to a certain operation mode, etc.). The first indication may comprise a system-specific identifier, identifying the local communication system, may be encrypted with a secret of the local communication system, etc.

The method may further comprise receiving a first response from the remote communication system to the first indication. The first response from the remote communication system may comprise the first data. For example, it may comprise the data in a format that it is readable by the implant without the requirement of any further data conversion (e.g., it may be arranged with the corresponding semantics). Therefore, the received response may fulfil the requirements of the communication protocol required by the implant and may simply be forwarded from the local communication system to the implant. The first response may not be readable/accessible by the local communication system. In particular, the semantics of the first data needs not be known or disclosed to the local communication system, such that it also cannot be illegally be derived from the local communication system, e.g., by hacker attacks, etc. Therefore, a secure end-to-end communication between implant and remote communication system may be enabled. Additionally or alternatively, the first response may comprise an authorization, etc.

The method may further comprise sending the first data to the implant upon receiving the first response, preferably in an encrypted manner, preferably encrypted with a secret of the implant. For example, the first data may be received from the remote communication system (e.g., in an encrypted manner, e.g., encrypted with a secret of the implant) and then simply be forwarded to the implant.

Sending the first data from the local communication system to the implant may facilitate a (secure) communication of the local communication system with the implant, e.g., by means of short-ranged communication technology (such as, e.g., near field communication (NFC), Bluetooth (preferably Bluetooth Low Energy (BLE)), Wi-Fi, etc.). This allows communication with the implant without requiring long-range communication capabilities (e.g., internet access, 5G, LTE, etc.) for the implant which may lead to security threats for the patient and which may arise from a direct communication of the remote communication system and the implant.

The secret of the implant may comprise a cryptographic key (also key hereinafter). Based at least in part on the cryptographic key, it may be possible that the encryption with a secret of the implant comprises a symmetric encryption (e.g., the remote communication system and the implant share the same cryptographic key). For example, the remote communication system and the implant may both be programmed with the secret at the time of manufacture of the implant (e.g., in a secure environment). It may also be possible that the encryption with a secret of the implant comprises an asymmetric encryption (e.g., the remote communication system and the implant share different cryptographic keys such as, e.g., a public key (e.g., applied to the first data to be encrypted) and a private key (e.g., used for decryption by the implant)). The secret may additionally or alternatively relate to a certificate which may be used to ensure the authenticity of the remote communication system when communicating with the implant and vice versa.

The first response may further include the first data in an encrypted manner, preferably encrypted with a secret of the implant. By including the first data in the first response in an encrypted manner, a secure communication of the first data from the remote communication system to the local communication system may be facilitated. Access to the data by unauthorized third parties may be prevented and data integrity (e.g., a prevention of unauthorized data changes) may be ensured. The encryption of the first data may in particular ensure a secure communication between the implant and the remote communication system via the local communication system (which may be understood as a relay device) and which may not be capable of accessing the data to be sent to the implant even in a scenario in which the local communication system has been hacked as the secret used to encrypt the first data may still be unknown for the local communication system, as it never leaves the implant and/or remote communication system (and/or since the semantics of the first data is not known to the local communication system). Therefore, by receiving the response in an encrypted manner, a highly secure end-to-end encryption may be facilitated between the remote communication device and the implant.

The secret of the implant may additionally or alternatively be chosen such that it ensures confidentiality (i.e., an absence of unauthorized data access by third parties) of the data to be sent to the implant.

Additionally or alternatively to the above steps, the method may comprise the following steps: receiving second data from the implant, and, without accessing the second data, transmitting a second indication associated with the second data to the remote communication system for processing.

By requiring that the second indication is forwarded to the remote communication system, the (secure) remote communication system may verify if the local communication device, from which the second indication has been forwarded, is authorized to receive and/or access the second data indicated (e.g., comprised at least in part) by the second indication. Thus, intrusion attacks by unauthorized third parties may be prevented.

The second data may relate to a response to an interrogation of the implant and/or to a programming command (e.g., confirming that the command has been implemented). The second indication may comprise a system-specific identifier, identifying the local communication system, may be encrypted with a secret of the local communication system, etc.

Receiving the second data may include receiving the second data in an encrypted manner, preferably encrypted with a secret of the implant.

Similar as outlined above with respect to the first data that may be sent from the remote communication system to the local communication system, also second data received from the implant (by the local communication system) may be encrypted to ensure data confidentiality and integrity during the communication. Also as outlined above, the encryption with a secret of the implant may ensure that the local communication system is only capable of relaying the received second data without being able to access/read the second data. Also concerning the second data, the local communication system does not need to know the semantics, such that the corresponding algorithms do not have to leave the implant and/or the remote communication system. The local communication system may, in the simplest case, just forward the second data to the remote communication system (which may use the same semantics and/or know the semantics used by the implant), possibly after appending further data and/or encryption with a secret of the local communication system, etc.

The secret used for the encryption of the second data may be identical to the secret used for the encryption of the first data (by the remote communication system). However, it may also be possible that different secrets are used for the first data and for the second data. In any case, it may be possible that the secrets discussed herein are static (e.g., the secret is a pre-determined OEM setting) which does not change over time. However, it may also be possible that the secret is dynamic (e.g., a new secret is generated for at least some of the communications (bi-directionally) between the remote communication system and the implant) which may provide an increased safety for the data to be communicated as successful man-in-the-middle attacks may be rendered even more seldom.

The method may further comprise receiving a second response from the remote communication system upon the second indication.

Similar to receiving a first response from the remote communication system to transmitting the first indication associated with the first data to the remote communication system (as outlined above), a second response from the remote communication system upon the second indication may only be received if the remote communication system has processed (e.g., (successfully) verified) the second indication. It may also be ensured that the local communication system cannot access or display information pertaining to the second data (due to its encryption and/or the unknown semantics), unless it receives the second response, such that it may be ensured that the received second data is of no use for unentitled local communication systems.

For that purpose, the second indication may include a system-specific identifier (associated with the local communication system). Additionally, it may also be possible that the second data, received from the implant, includes a system-specific identifier, indicating at least one local communication system which may be entitled to receive the second data. The remote communication system may only successfully verify the second indication if the system-specific identifier is among the at least one system-specific identifier comprised by the second data. Additionally or alternatively, the remote communication system may have access to a look-up table indicating the at least one local communication system which is entitled to communicate with the implant. The remote communication system may verify whether a system-specific identifier (received from the local communication system by means of the first and/or the second indication) is contained in the look-up table and thus eligible for communicating with the implant.

The second response may include the second data in a decrypted manner. Including the second data in the second response in a decrypted manner may be seen as the result of a successful verification (by the secure remote communication system) that the local communication system is entitled to receiving the second data. The decryption may relate to a decryption with respect to the secret shared between the remote communication system and the implant. The second data may however still be encrypted with a secret shared with the local communication system.

The method may further comprise providing a display based on the second response. For example, the display may be shown on a graphical user interface of the local communication device, e.g., a (touch-) screen. It is noted that it may be foreseen that the local communication system never accesses the second data, but only receives a second response based on which it may display aspects related to the second data. Additionally or alternatively, any other output (e.g., acoustic, voice, etc.) may be provided based on the second response. For example, the local communication system may not be able to access the second data, since it does not know its semantics. However, the remote communication system may provide the local communication system with a second response that includes data the local communication system is able to display and that reflects information conveyed in the second data (e.g., a heart rate measured by the implant, etc.). For example, the second response may comprise data to be displayed (e.g., at least a portion of the second data and/or data derived therefrom). For example, the display may relate to, e.g., a battery level of the implant, diagnostics (e.g., current health state, heart rate, electrocardiogram signal, effect of preceding settings sent to the implant, etc.), alerts (e.g., events endangering the health state of the patient), etc.

The display may, besides the local communication device, also be transmitted to a third device which is proven as trustful, such as, e.g., a hospital information system, a digital patient chart, an additional smart home device which is in communication with the local communication system etc.

The communication between the local communication system and the remote communication system may be encrypted, preferably with a secret of the local communication system.

By encrypting the communication between the local communication and the remote communication system, additional security measures may be provided such that the confidentiality and the integrity of the communication between the local communication system and the remote communication system is ensured and strengthened. This may in particular be valuable as the communication between the local communication device and the remote communication device may be performed over the internet (or any other public network).

The encryption of the communication with a secret of the local communication system may be performed in addition to an encryption with a secret shared between the remote communication system and the implant (which may be transparent to the local communication system).

Moreover, the encryption with a secret of the local communication system may also relate to a certificate issued for the local communication system. Therefore, the local communication system may identify itself as, e.g., an eligible local communication system for communication with the local communication system and the implant.

The present invention further relates to a local communication system for communication with an implant and with a remote communication system. The local communication system may comprise means for receiving a user input indicative of first data to be transmitted to the implant and means for transmitting a first indication associated with the user input to the remote communication system for processing, prior to sending the first data to the implant. Additionally or alternatively, the local communication system may comprise means for receiving second data from the implant, and means for transmitting, without accessing the second data, a second indication associated with the second data to the remote communication system for processing. The local communication system may additionally comprise means for performing any of the aforementioned method steps.

By implementing the above-mentioned functionality, the local communication system may be understood as remote control, interacting with the remote communication system, for controlling the implant in a secure manner. The local communication system may thus also be understood as a mediator in between the implant and the remote communication system, wherein the communication may be at least in part transparent such that the local communication system may be considered as a relay between both.

The present invention further relates to a method for communication, by a remote communication system, with a local communication system. The method may comprise the steps of: receiving, from the local communication system, a first indication indicative of a user input associated with first data to be transmitted to an implant, and sending a first response to the local communication system based at least in part on the first indication and stored data associated with the implant. The stored data associated with the implant may, e.g., comprise the secret of the implant which may be used to encrypt the data to be sent to implant. The stored data associated with the implant may additionally or alternatively comprise implant-specific information such as, e.g., an identifier and/or model of the implant, from which, e.g., semantics may be derived. By using said information, it may be possible that the remote communication system may support a variety of different implants. From the data associated with the implant it may also be derived whether the local communication system is entitled to communicate with the implant. Additionally or alternatively, it may also be possible that the first response is based on stored data relating to the local communication system, e.g., an expected checksum, a secret of the local communication system, etc. The above method may provide simple means to verify, by the remote communication system, whether or not the local communication system is entitled to communicate with the implant and/or whether it is specifically entitled to send the first data to the implant. For example, it may be possible that the stored data associated with the implant comprises information on which local communication system may be allowed to communicate with the implant, such that it may be prevented that any unauthorized local communication devices are capable of sending data to the implant.

The first response may further comprise the first data, preferably a command to be forwarded to the implant. The first response, first data and/or command may be created by the remote communication system based at least in part on the first indication received from the local communication system.

Additionally or alternatively, the first data, preferably the command, may be encrypted, preferably with a secret of the implant.

Additionally or alternatively, the method may comprise the steps of: receiving, from the local communication system, a second indication associated with second data that was received by the local communication system from an implant and sending a second response to the local communication system based at least in part on the second indication and stored data associated with the implant.

For example, the second response may include the second data in a decrypted manner.

With reference to a remote communication system, further method steps may be implemented which mirror the method steps outlined above for the local communication system. It may thus be ensured that the remote communication system is capable of serving any request from the local communication system and to initiate the local communication system to forward data to the implant.

The present invention further relates to a remote communication system for communication with a local communication system. The system may comprise means for receiving, from the local communication system, a first indication indicative of a user input associated with first data to be transmitted to an implant, and means for sending a first response to the local communication system based at least in part on the first indication and stored data associated with the implant. Additionally or alternatively, the remote communication system may comprise means for receiving, from the local communication system, a second indication associated with second data that was received by the local communication system from an implant and it may comprise means for sending a second response to the local communication system based at least in part on the second indication and stored data associated with the implant.

The remote communication system may comprise means for performing any of the aforementioned functionalities outlined herein with reference to method steps.

The remote communication system may be implemented as a (centralized and/or medical) cloud service. The cloud service may be administrated by the manufacturer of the implant and/or any other authorized third party supplier. The cloud service may also be part of a hospital information system. Such a logical organization of the remote communication system may provide the advantage of a centralized administration of authorized local communication systems (and their assignment to certain implants) by trained and experienced technical staff to ensure that only authorized communication with the implant are performed.

Moreover, the present invention relates to a method for communication, by an implant, with a local communication system. The method may comprise the step of receiving first data forwarded by the local communication system from a remote communication system, encrypted, by the remote communication system, with a secret shared between the remote communication system and the implant. Additionally or alternatively, the method may comprise the step of transmitting second data to the local communication system for forwarding to the remote communication system, encrypted, by the implant, with a secret shared between the implant and the remote communication system.

It may be foreseen that the communication capability of the implant requires a manual activation (e.g., by waking up, which can be done by means of a magnet and/or a coil placed close to the implant) prior to being able to receive and/or transmit data from/to the remote communication system via the local communication system. The manual activation may be achieved by placing a magnet (e.g., a bar magnet) close to the implant. The magnet may actuate a magnetic switch of the implant (by means of magnetic forces) and may thus put the implant in a pairing mode in which the implant may establish a communication with the local communication device. The pairing mode may, for example, only be activated for a limited amount of time. It may further be foreseen that the pairing mode may only be activated for a certain number of pairing processes per time interval (e.g., only once per hour, once per day, etc.). This may provide an additional security measure as a close physical contact to the patient is required prior to communicating with the implant. Therefore, any unauthorized remote programming of the implant may be prevented. An automatic deactivation of the pairing mode may also conserve battery power of the implant.

It may further be foreseen that the implant (automatically) returns to a safe and/or defined operation mode if an unexpected behavior occurs. Such an unexpected behavior may, e.g., relate to a loss of communication with the local communication system and/or between the local communication system and the remote communication system. The safe operation mode may be hardcoded on the implant. The implant may also be configured to enter the safe operation mode if any other unexpected event occurs.

Additionally, the present invention relates to an implant for communication with a local communication system. The implant may comprise means for receiving first data forwarded by the local communication system from a remote communication system, encrypted, by the remote communication system, with a secret shared between the remote communication system and the implant. Additionally or alternatively, the implant may comprise means adapted to transmit second data to the local communication system for forwarding to the remote communication system, encrypted, by the implant, with a secret shared between the implant and the remote communication system.

The implant may be a class III medical device. A class III medical device may relate to a device which directly connects with the (central) circulatory and/or nervous system. It may also be possible that the implant relates to any other medical implant.

The implant may be a cardiac rhythm management (CRM) device (e.g., a pacemaker, defibrillator), may be an implant for neuronal stimulations and/or any other (programmable and/or active) implant.

The present invention further relates to a system for communicating with an implant. The system may comprise the implant and/or the remote communication system and/or the local communication system as outlined herein.

For the user, participating in the communication between the remote communication system and the implant by means of the local communication system, the local communication system may be understood as the front end, whereas the remote communication system may be seen as the backend of communication with the implant.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The following figures are provided to support the understanding of the present invention:

FIGS. 1A-C Illustration of an overview of an exemplary encryption scenario between participating communication entities;

FIG. 2 Illustration of an overview of an exemplary setup of a system for communicating with an implant:

FIG. 3 Illustration of a communication diagram of an exemplary interrogation procedure of an implant:

FIG. 4 Illustration of a communication diagram of an exemplary editing and/or programming procedure of an implant:

FIG. 5 Illustration of a communication diagram of an exemplary transmission of a command to the implant:

FIG. 6 Illustration of a communication diagram of an exemplary IEGM data transfer.

### DETAILED DESCRIPTION

The following detailed description outlines possible exemplary embodiments of the present invention.

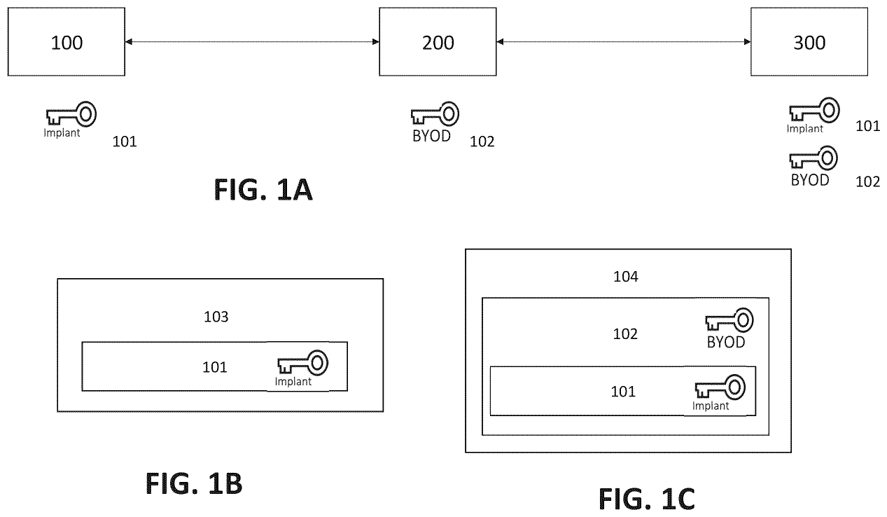

FIG. 1A shows an overview of an exemplary encrypted communication between a remote communication system 300, a local communication system 200 and an implant 100.

Implant 100 may be in direct communication with local communication system 200 but may not be in direct communication with remote communication system 300. Additionally, local communication system 200 may be in direct communication with remote communication system 300 and implant 100.

Any communication to (e.g., first data) and from (e.g., second data) implant 100 may be encrypted based at least in part on a secret 101 of implant 100. Secret 101 may be shared among implant 100 and remote communication system 300, but it may be unknown to local communication system 200.

Secret 101 may be shared between implant 100 and remote communication system 300 such that it ensures both confidentiality and integrity of the data communication between these two entities, e.g., by applying the Advanced Encryption Standard (AES) and/or Galois/Counter Mode (GCM). However, if implant 100 does not possess sufficient computation power and/or if implant 100 does not comprise a suitable additional dedicated cryptographic hardware module, a separate secret may be used to ensure confidentiality (e.g., AES and/or Rivest Cypher 5 (RC5)) and to ensure integrity (e.g., Cypher Message Authentication Code (CMAC)), respectively. When encrypting the data, it may be seen as preferable to first encrypt the data with respect to integrity and then with respect to confidentiality by applying the associated secrets in the respective encryption order. When decrypting the data, the decrypting order may follow a correspondingly inverted encryption order.

Secret 101 may, e.g., be obtained during the manufacturing process of implant 100 and may be disclosed to both implant 100 and remote communication system 300 (only). In particular, remote communication system 300 may store secret 101 for the entire lifecycle of implant 100. It may also be possible that implant 100 obtains more than one secret which is shared between implant 100 and remote communication system 300. In any case, the secret assigned to implant 100 is preferably individual (i.e., unique) to implant 100.

Local communication system 200 may forward any communication from/to implant 100 to/from remote communication system 300. Local communication system 200 may, for example, only relay the communication without being capable of semantically accessing the data which is relayed.

Local communication system 200 may additionally encrypt communications to remote communication system 300 with a secret 102 of the local communication system 200 (that may be a BYOD). It may also be possible that local communication system 200 adds additional local communication system-specific data (e.g., business layer protocol data, e.g., including one or more identifiers, certificates, and/or checksums, etc.) and/or remote communication system-specific data to the communication prior to encrypting the communication with secret 102. Said additional data may, e.g., be interpreted by remote communication system 300, and validity of the communications may be checked, based thereon. It may be foreseen that secret 102 is a unique secret 102 assigned to local communication system 200 which may be stored thereon (and on remote communication system 300). To ensure the security of secret 102 on local communication system 200, a Trusted Platform Module (TPM) may be provided on local communication system 200. Secret 102 may be for asymmetric encryption, e.g., based on Rivest-Shamir-Adleman (RSA). Secret 102 may ensure confidentiality and integrity of data as well as authenticity of the communication partner.

Additionally or alternatively, local communication system 200 may also receive communications from remote communication system 300 which may be encrypted with a secret 102 of the local communication system 200. When decrypting, by local communication system 200, local communication system 200 may check whether remote communication system 300 has added additional remote communication system-specific data (e.g., business layer protocol data) to the data to be sent to the local communication system 200. Said additional data may then be interpreted by local communication system 200, and validity of the communications may be checked, based thereon. Secret 102 may be stored by remote communication system 300.

Any of secrets 101 and/or 102 may refer to asymmetric and/or symmetric encryption.

FIG. 1B shows an exemplary illustration of the encryption of the communication sequence between implant 100 and local communication system 200 (as part of the communication between the implant 100 and the remote communication system 300). The communication between local communication system 200 and implant 100 may be encrypted with secret 101 that may be shared between implant and 100 and remote communication system 300 (but unknown to local communication system 200). The communication may further occur by means of a short-range communication technology. Such a short-ranged communication technology may be based on a technology-specific protocol 103 (e.g., Bluetooth (Low Energy)) which may foresee a technology-specific encryption for the underlying communication. The technology-specific protocol may be applied to the communication between local communication system 200 and implant 100 such that the already encrypted communication between implant 100 and local communication system 200 (as outlined above with respect to FIG. 1A) may additionally be wrapped by the technology-specific encryption 103 and may thus be seen as encrypted twice based on two different encryption protocols and/or secrets. It may be foreseen that a session specific secret (e.g., 102 or 103) is exchanged between implant 100 and local communication system 200 to additionally ensure the integrity and authenticity of the communication entities and/or that the technology-specific encryption may be session-specific.

FIG. 1C shows an exemplary illustration of the encryption of the communication sequence between the local communication system 200 and the remote communication system 300. The communication between the local communication system 200 and the remote communication system 300 may be encrypted with secret 101, and, optionally, additionally with a secret of the local communication system 102. The communication may be performed over the internet. The internet communication technology may possess its own communication protocol (e.g., HTTPS, SSL, SSH, etc.) which may also foresee a technology-specific encryption 104. Therefore, it may be possible that the encrypted communication between the local communication system 200 and the remote communication system 300 (as outlined above with respect to FIG. 1A) is additionally wrapped by the technology-specific protocol 104 and may thus be seen as encrypted two, or optionally three times based on three different encryption protocols and/or secrets.

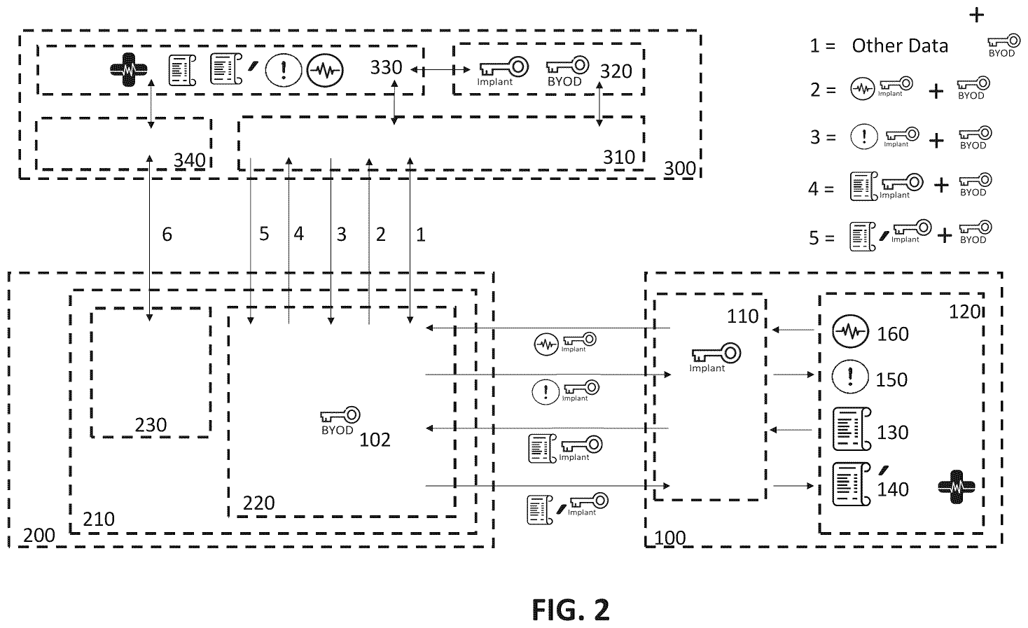

FIG. 2 shows an overview of a system as it may be used for the communication of a remote communication system 300 with an implant 100 by means of a local communication system 200 as described herein. It is noted that all aspects (e.g., the encryption scenarios and communication technologies) explained above with respect to FIG. 1 may also be applied to the aspects outlined below with respect to FIG. 2. In other words, any communication outlined below, may inherit the en- and decryption aspects explained with respect to FIG. 1.

Implant 100 may at least comprise a communication unit 110 and a therapy unit 120.

Communication unit 110 may refer to a logical entity of implant 100 which accounts for any communication related aspects (e.g., receiving and sending a communication (and associated data)) of implant 100. Communication unit 110 may contain a communication interface for performing a communication with local communication system 200. This may in particular comprise the application of the associated communication protocol of the communication technology. Communication unit 110 may additionally or alternatively comprise transmission and reception circuitry such as, e.g., one or more antennas. Additionally or alternatively, communication unit 110 may also comprise means for the encryption and decryption of data to be sent to the remote communication system 300 by means of the local communication system 200 as outlined above with respect to FIG. 1. Therefore, communication unit 110 may locally store respective secrets shared between implant 100 and remote communication system 300.

Communication unit 110 may internally communicate with therapy unit 120 and both units may be logically separated. Therapy unit 120 may comprise information on one or more therapy related aspects the implant is in charge of. Therapy unit 120 may thus be understood as administrating, e.g., real time data 160 associated with implant 100 such as, e.g., IEGMs (e.g., intracardiac ECGs), at least one therapy related command 150, such as, e.g., at least one of an activation or deactivation of temporary sub-programs (e.g., programs which should only be executed (temporarily) in addition to a main program being executed on the implant), an extension of at least one temporary sub-program (e.g., relating to rapid pacing for transcatheter aortic valve implantation), an activation or deactivation of an implantable cardioverter/defibrillator (ICD) therapy, an initiation of an emergency shock (e.g., in the sense of a defibrillator), an activation or deactivation of a VVI mode (e.g., the activation or deactivation of the stimulus of a single ventricle, e.g., to account for atrial fibrillation) and/or any generic administration of the implant (e.g., updates, downgrades, factory reset, etc.). Therapy unit 120 may further administrate direct access to the internal implant memory (e.g., comprising protocols, current settings, etc.). Direct access to the internal implant memory may be provided for at least two reasons: it may be possible that a dedicated access is provided to access data 130 to be sent from implant 100 by a dedicated communication link from therapy unit 120 to communication unit 110. Additionally or alternatively a (separate) access may be provided to store data 140 sent to the implant 100, e.g., by a dedicated communication link from communication unit 110 to therapy unit 120. It may thus be possible to provide different means for accessing the internal implant memory for up- and downstream data such that a load balancing and collision free communication with the implant is facilitated. The implant memory may, e.g., comprise therapy settings, statistics, recordings (e.g., of ECGs, in particular intracardiac ECGs), user data (e.g., owner of the implant, implantation date, etc.), firmware and/or other information. Therapy unit 120 may further be in internal communication with respective sensors which are capable of sensing health related aspects of the patient (e.g., a pulse rate, an ECG, in particular intracardiac ECGs, etc.).

Local communication system 200 may comprise an app 210. App 210 may comprise a communication unit 220 and a UI/UX unit 230 (e.g., as logical entities).

App 210 may be executed on local communication system 200 and may be downloadable from an app store and/or may be supplied by the manufacturer of the local communication system 200 (e.g., as an OEM software).

App 210 may provide functionality for facilitating the secure interrogation and/or programming of implant 100 by local communication system 200 via remote communication system 300. This goal may be provided by the capability for establishing a communication with implant 100, and registration and signing-in at the remote communication system 300. Further, data received from the implant 100 may be relayed to the remote communication system 300. App 210 (e.g., communication unit 220) may provide functionality for (bidirectional) data exchange 1 with remote communication system 300 (e.g., a communication unit 310 thereof), and/or for processing data (e.g., preparing its transmission and reception (e.g., by applying the underlying communication protocol)). Additionally or alternatively, it may be possible that app 210 (e.g., by a separate logical entity UI/UX unit 230) also provides means for data exchange with a front-end webservice 340 of remote communication system (as further described below), e.g., by receiving a user input by means of UI/UX unit 230 and forwarding of the received input to frontend webservice 340 of remote communication system 300.

App 210 may further be equipped with security measures to prevent reverse engineering, debugging, code, key and/or API manipulation. This may at least in part be based on app hardening mechanisms such as string encryption, class encryption, metadata removal, renaming, checksum(s), anti-debugging, jailbreak detection, shut-down and/or other suitable measures.

Additionally or alternatively, app 210 may also comprise measures to prevent the patient (carrying implant 100) from potentially dangerous situations (e.g., situations which are regarded as potentially harmful to the patient and/or which may be seen as life threating) which may arise from, e.g., erroneous configurations of implant 100 and/or unsuccessful communications with implant 100 and/or any other events. Such measures may, e.g., comprise commands (rules) to terminate the potentially harmful state and/or to provide diagnostics for a detailed understanding of the current situation and/or suggestions to be provided to the user of app 210 to overcome the current situation.

Communication unit 220 may refer to a (logical) entity of local communication system 200. Communication unit 220 may provide any software (e.g., protocols) and/or hardware (e.g., transmission and reception related circuitry such as, e.g., antennas) related aspects required for the communication of communication unit 220 with implant 100 and with remote communication system 300. Communication unit 220 may in particular provide functionality for at least two different communication technologies such that the communication between implant 100 and local communication system 200 may be different from the communication of local communication system 200 and remote communication system 300 (e.g., by using different communication technologies) as outlined above, e.g., with respect to FIGS. 1B and 1C. Communication unit 220 may comprise means for receiving and sending at least the data associated with reference numerals 140-160 as outlined above with respect to communication unit 110.

Communication unit 220 may provide encryption and decryption functionality such that any communication between the local communication system 200 and the remote communication system 300 may be en- and/or decrypted (cf. description of FIG. 1C above).

UI/UX unit 230 may be a logical entity separate from the communication unit 220. It may provide a user interface such that a user of local communication system 200 is capable of interacting with local communication system 200. By means of UI/UX unit 230, a graphical user interface (GUI) may be supplied to the user of local communication device 200 by means of, e.g., a touch sensitive screen to receive a user input. In such an application scenario, UI/UX unit 230 may further comprise a physical display and associated control hard- and software. By means of UI/UX unit 230 it may be possible to access webservices and/or apps (in addition or alternative to app 210) associated with implant 100 which may provide selection options of one or more parameters associated with settings for implant 100. UI/UX unit 230 may further comprise one or more of a microphone (for receiving voice commands and/or interrogations by the user, e.g., to be converted into a command for implant 100), a camera (e.g., for capturing gesture control related movements of a user), speakers, etc.

UI/UX unit 230 may be equipped with dedicated communication means (similar to the communication means disclosed with respect to communication unit 220) for communicating with remote communication system 300. It may also be foreseen that UI/UX unit 230 and communication unit 220 are logical entities which both use the service of underlying communication hard- and/or software to communicate with remote communication system 300. For example, UI/UX unit 230 and communication unit 220 may communicate by means of mutually independent data streams.

Remote communication system 300 may comprise a communication unit 310, a cryptographic key management system (CKMS) 320, a therapy unit 330 and a frontend webservice 340.

Communication unit 310 may refer to a logical entity of remote communication system 300 which accounts for any communication related aspects (e.g., receiving and transmission of a communication) of the remote communication system 300. Communication unit 310 may in particular be in communication with local communication system 200, e.g., communication unit 220. Communication unit 310 may comprise similar features as communication unit 220 such that a communication between communication unit 310 and communication unit 220 is facilitated. Communication unit 310 may not be capable of decrypting/encrypting any communications and may not interpret any implant related data-independent of the aspect whether the data is to be transmitted or received by remote communication system 300. Communication unit 310 may only check the validity of any local communication system-specific (see description for FIG. 1A above) data (e.g., business layer protocols, etc.) which may have been received.

However, in some examples, communication unit 310 may be capable of de- and encrypting data of local communication system 200 by means of secret 102, and it may perform the validity check after decryption with secret 102.

Communication between communication unit 220 and communication unit 310 may, e.g., comprise bidirectional communication 1 which may relate to non-medical data exchange between local communication system 200 and remote communication system 300. Such non-medical data may, e.g., comprise credentials associated with local communication system 200 to sign-in at remote communication system 300. Additionally or alternatively, the credentials may also be used to register local communication system 200 at the remote communication system 300 for the communication described herein. Communication 1 may further be used for the exchange of status information such as, e.g., availability information (e.g., current load factor with respect to processing capability and/or transmission bandwidth) of each of the local communication system 200 and the remote communication system 300. Additionally or alternatively, communication 1 may also relate to administration related data which may be used to update app 210 and/or to administrate all entitled local communication systems 200 for communicating with implant 100. The communication between communication unit 220 and communication unit 310 may be based on a wired (e.g., Ethernet, a fibre-based link, etc.) and/or a wireless communication technology (e.g., Wi-Fi, 5G (or successors), etc.). It is preferred to use a low-latency communication technology (with a latency<100 ms) allowing for data rates exceeding 100 Mbit/s (downstream).

The further uni-directional communications 2 and 3 may relate to the communication of (real time) data 160 and therapy related command(s) 150. Uni-directional communications 4 and 5 may relate to direct access to the internal memory of the implant and relate to data sent 130 from the implant 100 and to data to be sent 140 to the implant 100 to/from remote communication system 300.

Communication 1 may be encrypted with a secret 102 of the local communication system 200. Any of communications 2-5 may be encrypted with a secret 101 of the implant and may additionally be encrypted with a secret 102 of the local communication system 200.

Any of communications 2-5 may be encrypted twice or three times, e.g., with secret 101 and/or secret 102 as described in detail above with respect to FIG. 1C.

In any case, it may be required that local communication system 200 needs to sign in to remote communication system 300 prior to communicating. The process of signing-in may require a 2-factor authentication. Based thereon, remote communication system 300 may only offer access to local communication system 200 upon a reception and verification of the second factor.

It may be possible that any of said communications 1-5 between the local communication system 200 and the remote communication system 300 occurs without any further (relaying) entities in between or, e.g., by means of, e.g., at least one additional cloud system in between local communication system 200 and remote communication system 300.

However, it may also be possible that remote communication system 300 is operated in a distributed manner, e.g., by means of scalable real time IoT infrastructures such as cloud services, Software as a Service (Saas), etc. which may also be used to account for varying regional requirements. As an example, it may be possible that a remote communication system (such as remote communication system 300) needs to be operated in a country to serve a local communication system (such as communication system 200) also operated in that country. However, some countries may also allow that local communication systems operated in the country, may communicate with remote communication systems in other countries.

In any case, it is preferable but not necessarily required that remote communication system 300 is administrated by the manufacturer of implant 100 in a secure environment.

Communication unit 310 may be in internal communication with CKMS 320.

CKMS 320 may store the secret 101 shared between the remote communication system 300 and implant 100 and/or secret 102 shared between the remote communication system 300 and the local communication system 200. Secrets 101 and/or 102 may be securely stored in the CKMS 320 (which they may never leave) such that they do not appear in a public communication (e.g., in between the remote communication system 300 and the local communication system 200). Data stored in the CKMS may not be accessible by any other communication entity and/or third parties.

Additionally, CKMS unit 320 may also comprise means for decrypting data received from communication unit 310 using at least one of the stored secrets 101 and/or 102. Vice versa, CKMS may also use at least one of the stored secrets 101 and/or 102 for encrypting data to be transmitted, by remote communication system 300, to implant 100 and/or to local communication system 200.

In particular, CKMS unit 320 may receive data from communication unit 310 to be decrypted. CKMS 320 may then return decrypted data to communication unit 310. Vice versa, CKMS 320 may also receive data to be encrypted prior to an encrypted data transmission (e.g., by means of communication unit 310).

CKMS 320 may be in internal communication with therapy unit 330. Therapy unit 330 may receive decrypted data from CKMS unit 320 and may be seen as the final receiver of any data transmitted from implant 100. Vice versa, therapy unit 330 may also forward data to be transmitted to implant 100 and/or local communication system 200, to CKMS 320 for encryption by the respective secret.

Therapy unit 330 may comprise means for processing received data, e.g., from implant 100 (e.g., to decide whether the implant improves the health state of the patient carrying implant 100 or whether a deterioration of the health state of the patient carrying implant 100 occurs). Therapy unit 330 may further comprise means for eliciting whether received data may be presented to local communication system 200 (as part of a validity check). If it is determined that the received data may be presented to local communication system 200, associated data may be forwarded to frontend webservice 340 with which therapy unit 330 is in internal communication. It may also be possible, vice versa, that therapy unit 330 receives an indication of data from frontend webservice 340 which is to be transmitted, by remote communication system 300, to implant 100. In that case, therapy unit 330 may elicit whether the local communication system 200 and/or the respective data is entitled to be transmitted to implant 100. This may relate to a validity check as disclosed herein. If so, the corresponding data may be generated and then encrypted with secret 101 by CKMS 320.

Similarly as UI/UX unit 230 and communication unit 220, frontend webservice 340 and communication unit 310 may relate to separate logical entities.

It may further be foreseen that therapy unit 330 comprises means for storing and/or administrating patient related data such as, e.g., the assignment of certain implants to patients (e.g., by means of an implant specific ID assigned to a patient), one or more recordings of health related data associated with a patient (e.g., realtime IEGMs, therapy settings, statistics, historic readouts of the internal memory of the implant) and/or a set of commands to be sent to implant 100. This data may be stored in remote communication system 300, e.g., in a database, a blockchain, in text files, etc. Therapy unit 330 may be seen as the only location at which health related data may be found in a decrypted manner (besides implant 100).

Moreover, therapy unit 330 may comprise a detailed knowledge of the setup and/or memory structure of implant 100. Therefore, any (first) data to be sent to implant 100, by remote communication system 300, which may most likely not follow the syntax requirements of implant 100, may be translated, by therapy unit 330, into a command/data which may be understood by implant 100 (e.g., in response to a first indication received by remote communication system 300 from local communication system 200, in particular received by webservice 340 from UI/UX unit 230). Vice versa, if therapy unit 330 receives (second) data from implant 100, therapy unit 330 may translate the received data (following the syntax of implant 100) into a format which may be understood by other entities of remote communication system 300, e.g., frontend webservice 340 with which therapy unit 330 is in internal communication. Frontend webservice 340 may then provide a response to UI/UX unit 230 such that the latter may display aspects of the received data.

Additionally it may also be possible that therapy unit 330 is equipped with one or more measures to prevent the patient (carrying implant 100) from events which may deteriorate the health state of the patient and/or which may even be seen as life threating similar as outlined above with respect to app 210.

Frontend webservice 340 may provide a webserver which may be accessible over the intranet and/or internet. Frontend webservice 340 may be understood as an interface for data exchange between the remote communication system 300 and local communication system 200. Frontend webservice 340 may comprise a webserver and may comprise a website. Data received, by remote communication system 300, and processed therein as described above, may be displayed by means of frontend webservice 340. The display may be based on providing a website which may be accessed by an entitled entity, e.g., UI/UX unit 230. The display may, e.g., be based on providing charts of temporal evolutions of vital parameters associated with a patient carrying implant 100. Additionally or alternatively, webservice 340 may provide means for selecting (e.g., buttons, sliders, toggle switches, check boxes, etc.) certain operation parameters implant 100 should be set to. Alternatively or additionally, it may also be possible that frontend webservice 340 relates to certain sockets, an SSH tunnel, etc. by means of which the respective data may be accessed by entitled entities.

The website may be server-sided or client-based. It may also be possible that the website comprises a combination of both technologies.

Any input received by frontend webservice 340, e.g., from UI/UX unit 230, may be provided to therapy unit 330 for further processing as described above. However, it may also be possible that a user input is associated with data that is not required to be forwarded to implant 100 (e.g., an adjustment of the scale of a diagram). In such a case, the data associated with the user input may solely be processed by frontend webservice 340. However, it may also be possible that the received input undergoes a validity check in therapy unit 330 prior to being returned to frontend webservice 340 which may update the associated display.

It may further be possible that frontend webservice 340 also comprises one or more measures to prevent any events which may be harmful for the health state of the patient as outlined above with respect to therapy unit 330.

An entitled entity to access frontend webservice 340 may, e.g., be local communication system 200 and in particular UI/UX unit 230 as described above. UI/UX unit 230 may, for example, comprise an internet browser for accessing the website supplied by frontend webservice 340. Frontend webservice 340 may be accessed by UI/UX unit 230 by means of (bidirectional) communication 6. Communication 6 may also be encrypted and independent of communications 1-5 performed by the communication unit 220 and communication unit 310.

Communication 6 between UI/UX unit 230 and remote communication system 300 may be logically separated from the communication by communication unit 220 with remote communication unit 300. The communication technology used by UI/UX unit 230 may differ from the communication technology used by communication unit 220 or may be equal to it.

Communication 6 may preferably be encrypted with an asymmetric encryption technology such as RSA to ensure the confidentiality, integrity and authenticity (e.g., of the communicating entities) of the data exchanged between frontend webservice 340 and UI/UX unit 230. Methods to generate and exchange a respective secret/key may, e.g., be the Hypertext Transfer Protocol Secure (HTTPS) and/or Message Queuing Telemetry Transport (MQTT). To avoid vulnerability for man-in-the-middle (MITM) attacks and to ensure that UI/UX unit 230 only communicates with the right frontend webservice 340 and not with any third-party webservices which mimic to be frontend webservice 340, HTTP Public Key Pinning (HPKP)/SSL-Pinning and/or any other suitable technology may be used to ensure that only frontend webservice 340 may acquire a unique and trustful certificate with which frontend webservice 340 may authenticate itself to UI/UX unit 230 as trustful.

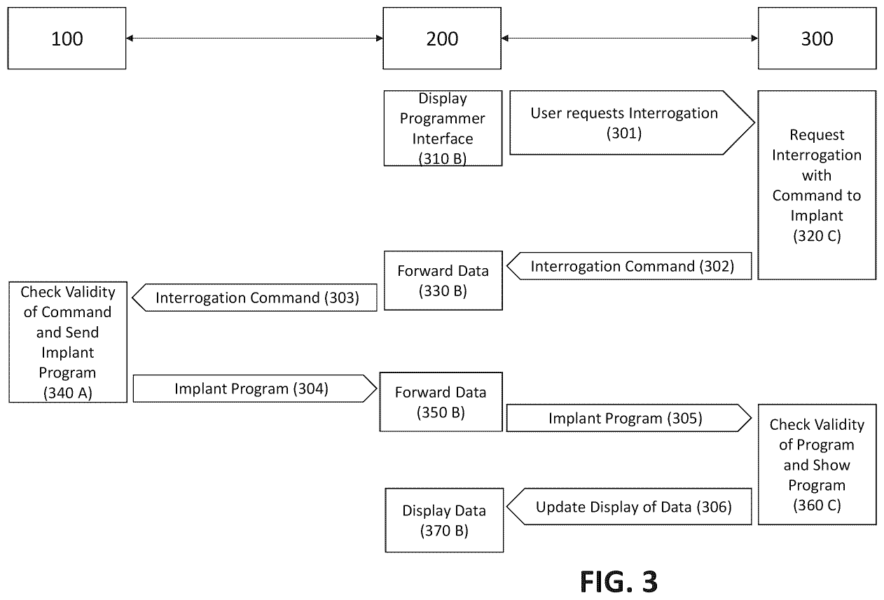

The following FIGS. 3-6 illustrate exemplary communication diagrams of communications between implant 100, local communication system 200 and remote communication system 300. The communication entities described therein may inherit the properties of the communication entities and interrelated communication methods (e.g., de- and encryption sequences of FIGS. 1A-C) as described above with reference to FIGS. 1A-C and FIG. 2. The communication diagrams disclosed below may not be seen as exclusive communication sequences. Instead, the present invention also allows for additional communication sequences which are not expressly described herein.

FIG. 3 shows a communication diagram for an exemplary interrogation of implant 100.

Local communication system 200 may display a programmer interface 310 B to a user of local communication system 200 (e.g., via UI/UX unit 230). The user may provide a corresponding user input, and local communication system 200 may receive the input of the user, e.g., an input associated with a request for interrogating implant 100.

Local communication system 200 may send the input (e.g., request for interrogation 301), e.g., in an indication associated with the user input (first indication associated with first data to be sent to implant 100, wherein the first data would be interrogation command 302, cf. below) to remote communication system 300. Remote communication system 300 may receive the request for interrogation 301 and may verify the request (as explained above with respect to therapy unit 330). If the verification is successful, remote communication device 300 may create and/or select a command 320 C for the implant (i.e., a command with an implant-specific syntax) to request the interrogation. The command 320 C may be encrypted with a secret of the implant.

Remote communication device 300 may then send a response including interrogation command 302 to local communication system 200. Local communication system 200 acts as a relay device and may forward 330 B the data associated with interrogation command 302. The data forwarded 330 B may be received as interrogation command 303 by implant 100.

Implant 100 may check the validity of the received command 340 A. If the validity check is successful (e.g., the command comprises parameters within certain parameter ranges, is plausible, comprises an authorized command, etc.), the received command may be executed by the internal implant logic and data on the implant program 304 may be sent to local communication device 200 (which may be seen as second data, as outlined herein).

Local communication device may forward 350 B the data on implant program 304 received from implant 100 (e.g., it may send a notification comprising the data, possibly further encrypted or appended with further data, as outlined herein). The forwarded data may be received by remote communication system 300. Remote communication system 300 may receive the data associated with implant program 305 and may perform a validity check 360 C.

If the validity check 360 C is successful, remote communication system 300 may provide information associated with the data on implant program 305 to local communication system 200 (second response) by means of, e.g., frontend webservice 340. This may cause an update of the display of data 306 (e.g., by modifying the source code of a website by means of which the local communication system 200 accesses the data) such that a display of the requested data 370 B (i.e., the current implant program) is displayed on the local communication system 200.

For the sake of safety, it may be foreseen that the user of local communication system 200 is required to be in charge with a working offline defibrillator (e.g., a classical defibrillator known from hospitals, train stations, offices, etc.) when conducting any interrogations and/or programming of implant 100 to allow immediate reactions if an unexpected event occurs which may be life threating as the interrogation and/or programming of implant 100 may only be possible if implant 100 is in communication with remote communication system 300 (by means of local communication system 200).

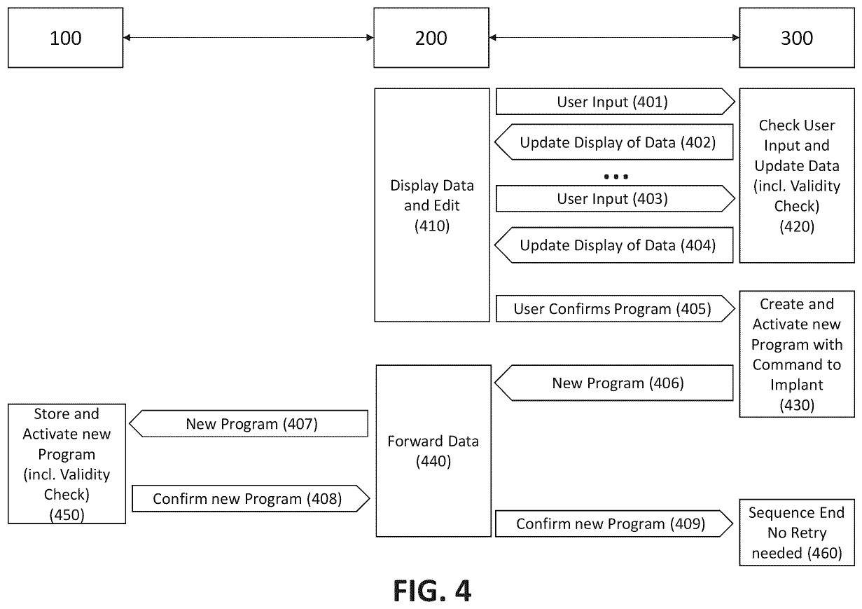

FIG. 4 shows a communication diagram for an exemplary editing/programming of implant 100.

An editing and/or programming of local communication system 200 may be initiated by an interaction of a user of local communication system 200 (e.g., by means of UI/UX unit 230).

However, it may additionally or alternatively be possible, that App 200 automatically generates a request (similar to a user input) which may be received by UI/UX unit 230. UI/UX unit 230 may generally display data and allow editing data 410. A corresponding user input 401 may be received which may be transmitted to remote communication system 300.

Remote communication unit 300 may check 420 the user input which may comprise a validity check (as outlined above). If the validity check is successful, data associated with the user input may internally be updated on the remote communication device 300 (e.g., stored in the therapy unit 330 as discussed above).

It may be possible that the update of internal data of remote communication system 300 may require an update of the data displayed 402 to the user (e.g., by means of an update of content displayed by frontend webservice 340 and an access of the content, e.g., by means of UI/UX unit 330) such as, e.g., moving a slidable switch to a new/updated position.

Data may then be displayed 410 and (re-)edited based on the updated data on local communication system 200. One or more additional user inputs (exemplarily represented by user input 403) may be transmitted to remote communication device 300 which may perform a validity 420 check for each of the user inputs 403, followed by a potential update of data as it has been explained beforehand. An update of the display of data 404 may be followed.

This procedure may be performed in an iterative loop until a user confirms a certain program (e.g., a set of desired operation parameters to be transmitted to implant 100). A corresponding (first) indication associated with the corresponding first data (here: confirming the program) to be sent to implant 100 may then be sent to remote communication system 300. In the shown example, indication comprises the confirmation of the program 405.

Based at least in part on the indication 405, remote communication system 300 may create 430 a new program (e.g., the byte code required for executing the desired program) comprising at least one command to be sent to implant 100. In other words, the program (e.g., corresponding data) to be sent to implant 100 may be created by remote communication system 300 based at least in part on the confirmed program 405 by the user. The new program may either relate to the main program being executed on implant 100 (and/or effectively a parameter change of the main program which requires a corresponding update of change of the main program) and/or may relate to a sub-program which is to be executed on implant 100 in addition or alternatively to the main program. The new program may, e.g., relate to a (temporary) sub-program which may be terminated automatically (e.g., after a certain pre-defined amount of time) and/or in reaction to an external trigger (e.g., if a certain pulse rate of the associated patient is achieved) and/or any other event.

The new program 406 may then be sent to local communication system 200, e.g., in a first response. Local communication system 200 may forward 440 the new program 407 (first data) to implant 100.

Implant 100 may receive new program 407 and may store 450 new/received program. As part of the activation, implant 100 may also perform a validity check (e.g., the program operates within certain parameter ranges and or bounds, is plausible, comprises authorized programming fragments and/or commands, etc.).

If the validity check of implant 100 is successful, the new program may be activated and a confirmation of the new program 408 may be sent to local communication system 200 (second data).

Said confirmation of the new program 408 may be understood as a handshake transmission. It is noted that any communication between two devices described herein may require a handshake mechanism (e.g., by transmitting respective ACK/NACK messages) to ensure a successful communication. In case a communication turns out to be not successful (e.g. the handshake signal carries an indication that the new program (or any other indication) has not been successfully received), a retransmission of said new program (or any other communication) may occur (either immediately and/or after a certain amount of time). It may also be possible that the new program is resent once (if an unsuccessful handshake is indicated) or that the new program (or any other communication) is resent for a certain number of times (depending on the context of the communication).

Local communication system 200 may forward the new program 409, e.g., in a corresponding second indication.

The forwarded 440 data may be received by remote communication system 300 as a confirmation 409 of the new program. The reception of the confirmation of the new program 409 may be interpreted as the sequence end 460 (i.e., an end of iterating steps 401-404) and that no retry of the sequence is needed.

It may be possible that an additional confirmation of sequence end 460 is transmitted to local communication system 200 (second response).

In any communication sequence, it may also be possible that certain settings of implant 100 (e.g., an active sub-program and/or single commands sent to implant 100) cause a state which may potentially be regarded as harmful for the patient. To overcome such a scenario, implant 100 (and/or remote communication system 300) may foresee measures for reducing the risk for the patient which may arise from an unsecure state. An unsecure state may, e.g., arise from unexpected terminations of a communication (e.g., in between implant 100, local communication system 200 and/or remote communication system 300), it may arise if certain sub-systems (e.g., any of the entities which described above with respect to FIG. 2) are currently not available. Additionally or alternatively, it may also be possible that such an unsecure state occurs because of a processing error (e.g., if a command sent to implant 100 is interpreted in an erroneous manner by implant 100 (e.g., if the syntax associated with such a command is erroneous) and/or if errors during the validity check occur. Additionally or alternatively, it may also be possible that an unsecure state results if a communication and/or processing timeout occurs. While generally security measures are taken to avoid such states, it is nevertheless desirable to foresee the mentioned measures to reduce risk in such a state, particularly for class III implants. If such a state occurs, a command (e.g., sent to implant 100 by remote communication system 300) may not be confirmed by implant 100 within a pre-defined time interval and/or a program sent to implant 100 may not be executed within a pre-defined time interval. It may also be possible, that an unsecure state results upon an exceeding of a pre-defined number of retransmissions of a certain command (e.g., upon an indication by a handshake signal) that a command has not safely been received and/or in scenarios in which a validity check has not successfully been executed for a certain pre-defined number of iterations.

Such security measures may comprise one or more sub-programs which may be (temporarily) activated to overcome a current potential risk (i.e., for the health state of the patient) arising from the unsecure state. Additionally or alternatively, it may also be foreseen that a current (active) sub-program (which may be responsible for the unsecure state) may be stopped and the implant 100 may return to executing a well-defined main program only. A similar procedure may be executed if it is determined that a connection between any of the communicating entities (implant 100, local communication system 200, remote communication system 300) is unexpectedly terminated.

If an unsecure state is detected (e.g., upon sending new program 407) to implant 100, new program 407 may be deleted and the programming process may be restarted. It may also be possible that implant 100 (alone and/or in communication with remote communication system 300) may automatically return to a safe state, e.g., by applying one or more of the above-mentioned security measures.

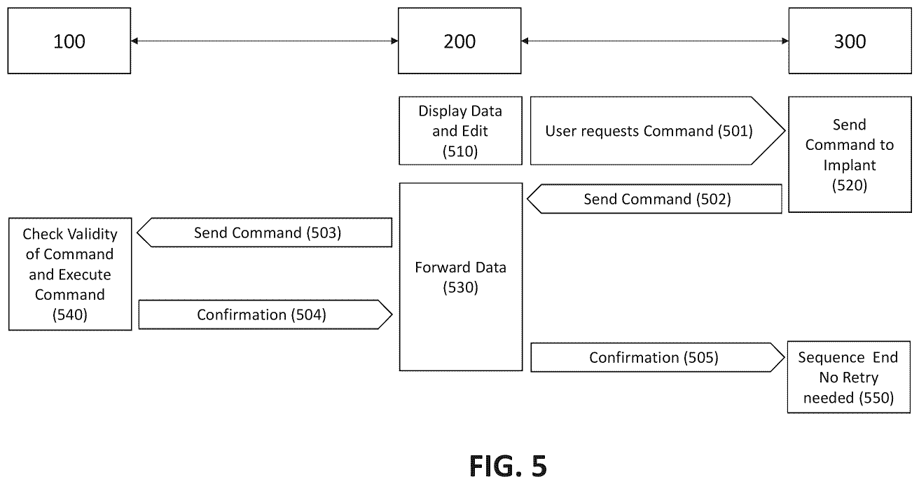

FIG. 5 shows a communication diagram for an exemplary transmission of a command to implant 100.

Local communication system 200, e.g., UI/UX unit 230, may display 510 data and may provide means for editing data as described above with respect to FIG. 4. A user may provide user input which indicates a request 501 for a command (e.g., a user may select a request from a list of pre-defined requests and/or the user may type in a certain request; e.g., via UI/UX unit 230) which request may be sent to remote communication system 300, e.g., in a first indication. The request may be indicative of the command (first data) to be sent to implant 100.

Remote communication system 300 may perform a validity check (as outlined above with respect to FIGS. 2-4) based at least in part on the request. If the validity check is successful, remote communication system 300 may create and/or select the respective command and may initiate the sending 520 of the respective command to implant 100. The command may be in a format understood by implant 100 but not by local communication system 200.

The command may be sent 502 to local communication system 200, e.g., in a first response. Local communication system 200 may forward 530 the corresponding data and send 503 the command to implant 100.

The received command may undergo a validity check 540 by implant 100. If the validity check is successful, implant 100 may execute the command.

Implant 100 may send a confirmation 504 to local communication system 200 (e.g., a handshake signal as described above) (second data). Local communication system 200 may forward 530 the associated data (second indication).

More specifically, local communication system 200 may send confirmation 505 to remote communication system 300. The reception of confirmation 505 may be interpreted as the sequence end 550 (i.e., a successful execution of the requested command) and that no retry of the sequence is needed.

A confirmation of the sequence end 550 may be transmitted to local communication system 200 (second response).

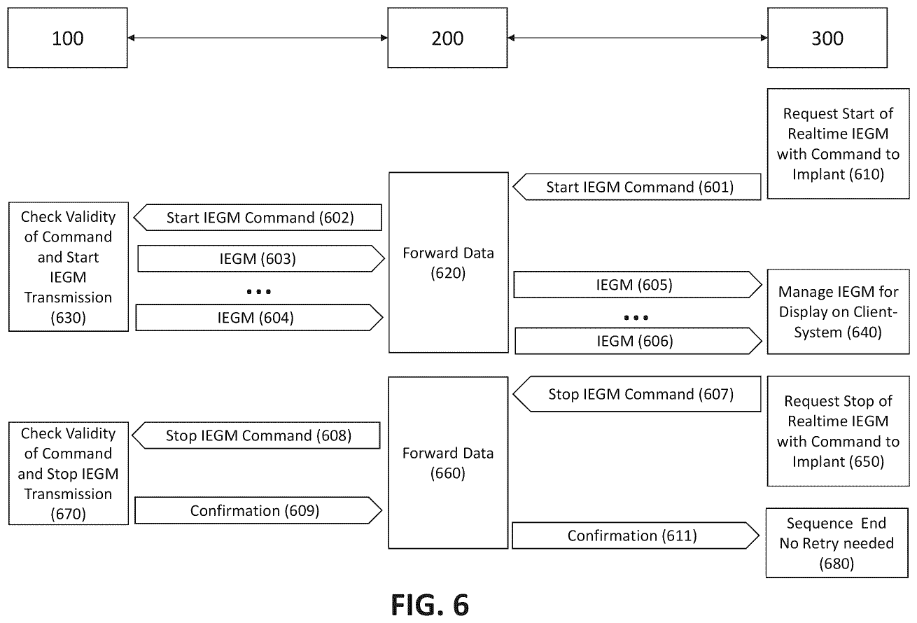

FIG. 6 shows a communication diagram for an exemplary IEGM data transfer.

An IEGM data transfer may be initiated by local communication system 200 (e.g., based on the transmission of a respective request to remote communication system 300, similarly as, e.g., discussed above with respect to FIG. 3 (user requests Interrogation (301)) but not shown in FIG. 6, e.g., in a first indication

The remote communication system 300 may receive the request and may perform a validity check. If the validity check is successful, remote communication system 300 may create 610 a request to start a realtime IEGM (first data) by means of a respective command to be sent to implant 100.

Alternatively, it may also be possible that the creation 610 of a request to start a realtime IEGM by means of a respective command to be sent to implant 100 is initiated by remote communication system 300 without an external input.

In any case, remote communication system 300 may send a start IEGM command 601 to local communication system 200, e.g., in a first response.

Local communication system 200 may transmit a start IEGM command 602 to implant 100 (first data).

Upon receiving start IEGM command 602, implant 100 may check 630 the validity of the received command and may start the IEGM transmission if the validity check is successful. The validity check at implant 100 may be performed as described above with reference to FIGS. 2 to 5.

If the validity check is successful, implant 100 may start IEGM transmission by, e.g., sending one or more IEGMs (603, 604) to local communication system 200 (second data). A single IEGM may, e.g., relate to a certain temporal sequence (e.g., Is, 1 min, 10 min, etc.) of IEGM related data. It may be possible that the IEGMs are generated such that IEGMs 603 and 604 may be concatenated to obtain a continuous IEGM recording. Additionally or alternatively, it may also be possible that IEGMs 603 and 604 map discrete temporal bins (e.g., 1 s, 1 min, 1 hour long capture of IEGM related data which may be intermittent by a temporal gap which may, e.g., comprise a 1 s, 1 min, 1 hour, etc. period during which no IEGM related data is recorded) of IEGM data.

Local communication system 200 may forward 620 the associated data (without accessing the data). Local communication system 200 may thus send the one or more IEGM as IEGM 605 and/or 606 to remote communication system 300, e.g., in corresponding second indications.

Remote communication system 300 may receive the one or more IEGM and may manage/process the received one or more IEGM to be displayed to local communication system 200. The processing may comprise performing a validity check as described above with respect to FIGS. 3 to 5 for scenarios in which remote communication system 300 receives data from local communication system 200.

The managing 640 may further comprise internally forwarding, at the remote communication system 300, of the associated data to frontend webservice 340 for providing a display to local communication system 200. This may include forwarding display information including or being based on at least a portion of the associated data, e.g., in a second response. Local communication system may access the information by means of UX/UI unit 230 interacting with frontend webservice 340, as, e.g., described above with respect to FIG. 2.

It is preferred that the above mentioned method steps are performed such that UX/UI unit 230 of local communication system 200 continuously receives IEGMs such that a user of local communication system 200 may have access to realtime IEGM data such that a user may, e.g., track the temporal evolution of the IEGM data.

It may be foreseen that IEGMs are created by implant 100 until a stop command is received. Requesting 650 to stop realtime IEGM with a respective command may be initiated by a user of local communication system 200, similar to the requesting the start of realtime IEGM as outlined above.

Alternatively or additionally, it may also be foreseen that remote communication system 300 initiates the stop of the creation of realtime IEGM. The initiation by remote communication system 300 may occur after, e.g., a certain period of time has elapsed (e.g., after 1 min, after 10 min, after 1 hour, etc.). Alternatively or additionally, it may also be possible that the stop of the creation of IEGMs is initiated upon leaving app 210 on local communication system 200. In such a case, the initiation of a stop of the creation of IEGMs may be understood as a secure termination of the communication of remote communication system 300 and implant 100 via local communication system 200. Additionally or alternatively, it may also be foreseen that the initiation of a stop of the creation of IEGMs may be initiated upon disconnection of the connection between any of the implant 100, the local communication system 200 and/or the remote communication system 300. Such a disconnection may, e.g., arise from a low signal strength, e.g., if the communication between the local communication system 200 and the remote communication system 300 is based on a 5G connection.

In any of said scenarios, remote communication system may create a command requesting 650 to stop realtime IEGM, wherein the command is to be sent to implant 100.

Remote communication system 300 may send the stop IEGM command 607 to local communication system 200, e.g., in a response.

Local communication system 200 may forward 660 the associated data (e.g., without accessing the data). Local communication system 200 may thus send a stop IEGM command 608 to implant 100.

Implant 100 may receive stop IEGM command 608 and may check 670 the validity of the command. The validity check may be implemented identically to the validity check as described above with reference to reference numeral 630.

If the validity check is successful, implant 100 may stop the IEGM transmission. Implant 100 may send a confirmation 609 to local communication system 200 to indicate that the transmission of IEGM has successfully been stopped.

Local communication system 200 may forward 660 the associated data. Local communication system 200 may thus send a confirmation 611 to remote communication system 300.

Confirmation 611 may be received by remote communication system 300 and may be interpreted as sequence end 680 (i.e., an end of IEGM transmission) and that no retry of the sequence is needed.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Method for communication, by a local communication system, with an implant and with a remote communication system (300), comprising:
receiving a user input indicative of first data to be transmitted to the implant,
transmitting a first indication associated with the user input to the remote communication system for processing, prior to sending the first data to the implant; and/or
receiving second data from the implant,
without accessing the second data, transmitting a second indication associated with the second data to the remote communication system for processing.

2. Method of claim 1, further comprising receiving a first response from the remote communication system to the first indication.

3. Method of claim 2, further comprising sending the first data to the implant upon receiving the first response, preferably in an encrypted manner, preferably encrypted with a secret of the implant.

4. Method of claim 2, wherein the first response includes the first data in an encrypted manner, preferably encrypted with a secret of the implant.

5. Method of claim 1, wherein the receiving the second data includes receiving the second data in an encrypted manner, preferably encrypted with a secret of the implant.

6. Method of claim 1, further comprising receiving a second response from the remote communication system upon the second indication.

7. Method of claim 6, wherein the second response includes the second data in a decrypted manner.

8. Method of claim 6, further comprising providing a display based on the second response.

9. Method according to claim 1, wherein communication between the local communication system and the remote communication system is encrypted, preferably with a secret of the local communication system.

10. A local communication system for communication with an implant and with a remote communication system, comprising:
means for receiving a user input indicative of first data to be transmitted to the implant;
means for transmitting a first indication associated with the user input to the remote communication system for processing, prior to sending the first data to the implant; and/or
means for receiving second data from the implant;
means for transmitting, without accessing the second data, a second indication associated with the second data to the remote communication system for processing.

11. Method for communication, by a remote communication system, with a local communication system, comprising:
receiving, from the local communication system, a first indication indicative of a user input associated with first data to be transmitted to an implant,
sending a first response to the local communication system based at least in part on the first indication and stored data associated with the implant; and/or
receiving, from the local communication system, a second indication associated with second data that was received by the local communication system from an implant;
sending a second response to the local communication system based at least in part on the second indication and stored data associated with the implant.

12. Method according to claim 11, wherein the first response comprises the first data, preferably a command to be forwarded to the implant.

13. Method according to claim 12, wherein the first data, preferably the command, is encrypted, preferably with a secret of the implant.

14. A remote communication system for communication with a local communication system, comprising:
means for receiving, from the local communication system, a first indication indicative of a user input associated with first data to be transmitted to an implant;
means for sending a first response to the local communication system based at least in part on the first indication and stored data associated with the implant; and/or
means for receiving, from the local communication system, a second indication associated with second data that was received by the local communication system from an implant;
means for sending a second response to the local communication system based at least in part on the second indication and stored data associated with the implant.

15. System for communicating with an implant, comprising at least two of the following:
an implant;
a remote communication system for communication with a local communication system, the remote communication system comprising:
means for receiving, from the local communication system, a first indication indicative of a user input associated with first data to be transmitted to the implant;
means for sending a first response to the local communication system based at least in part on the first indication and stored data associated with the implant; and/or
means for receiving, from the local communication system, a second indication associated with second data that was received by the local communication system from the implant;
means for sending a second response to the local communication system based at least in part on the second indication and stored data associated with the implant; and
the local communication system for communication with the implant and with the remote communication system, comprising:
means for receiving a user input indicative of first data to be transmitted to the implant;
means for transmitting a first indication associated with the user input to the remote communication system for processing, prior to sending the first data to the implant; and/or
means for receiving second data from the implant,
means for transmitting, without accessing the second data, a second indication associated with the second data to the remote communication system for processing.
